(19) United States
(12) Patent Application Publication
(10) Pub. No.: US 2022/0073754 A1
(43) Pub. Date: Mar. 10, 2022

(54) COATED MEDICAL DEVICE PRODUCT AND PROCESS

(71) Applicant: SILCOTEK CORP., Bellefonte, PA (US)

(72) Inventors: Patrick DICK, Altoona, PA (US); David A. SMITH, Bellefonte, PA (US); Jesse BISCHOF, Bellefonte, PA (US); Ricky EDMISTON, State College, PA (US)

(21) Appl. No.: 17/407,902

(22) Filed: Aug. 20, 2021

Related U.S. Application Data
(60) Provisional application No. 63/074,859, filed on Sep. 4, 2020.

(51) Int. Cl. C09D 5/00; A61L 27/28; A61L 29/08; A61L 31/08

(52) U.S. Cl. CPC C09D 5/00; A61L 27/28; A61L 2400/18; A61L 31/08; A61L 2420/00; A61L 29/08

(57) ABSTRACT

Medical device products have a coating with NAMSA Class VI certification and properties corresponding to the prior art coatings within U.S. Pat. No. 10,604,660. Medical device processes use the medical device product having a coating with NAMSA Class VI certification and properties corresponding to the prior art coatings within U.S. Pat. No. 10,604,660.

### PRIORITY

The present application is a non-provisional patent application claiming priority and benefit of U.S. Provisional Patent Application No. 63/074,859, filed Sep. 4, 2020 and entitled “COATED MEDICAL DEVICE PRODUCT AND PROCESS,” the entirety of which is incorporated by reference.

### FIELD OF THE INVENTION

The present invention is directed to medical device products and processes using such medical devices. More particularly, the present invention is directed to such medical devices and processes requiring properties corresponding with NAMSA Class VI certification.

### BACKGROUND OF THE INVENTION

NAMSA Class VI certification provides support for materials being biocompatible. Materials, such as certain parylene thin films and coatings are known to have such certifications. However, only alkylated parylenes are amorphous, thereby limiting applications. As a result of the coatings being semi-crystalline they scatter light and have limitations of use. Certain parylenes melt at temperatures of less than 300° C. Others anneal at temperatures of 400° C. and then degrade.

Coated medical device products and processes able to operate with expanded operating conditions would be desirable in the art.

### BRIEF DESCRIPTION OF THE INVENTION

In an embodiment, a medical device product has a coating with NAMSA Class VI certification and properties corresponding to the prior art coatings within U.S. Pat. No. 10,604,660.

In an embodiment, a medical device process uses a medical device product having a coating with NAMSA Class VI certification and properties corresponding to the prior art coatings within U.S. Pat. No. 10,604,660.

Other features and advantages of the present invention will be apparent from the following more detailed description, taken in conjunction with the accompanying drawings which illustrate, by way of example, the principles of the invention.

### DETAILED DESCRIPTION OF THE INVENTION

Provided are medical device products having a coating with NAMSA Class VI certification and properties corresponding to the prior art coatings within U.S. Pat. No. 10,604,660, which is hereby incorporated by reference. In addition, provided are medical device processes using such medical device products.

The Dursan® process, exclusively available from SilcoTek Corporation in the United States, was improperly identified as being incompatible with properties corresponding with NAMSA Class VI certification. Likewise, coatings disclosed in U.S. Pat. No. 10,604,660, entitled “WEAR RESISTANT COATING, ARTICLE, AND METHOD,” which is hereby incorporated by reference in its entirety, were improperly identified as being incompatible with NAMSA Class VI certification.

According to embodiments of the disclosure, the medical device product includes properties within the scope of U.S. Pat. No. 10,604,660, for example, having a carbon, hydrogen, oxygen, silicon in an amorphous arrangement. Suitable products include, but are not limited to, components capable of being produced into a coated article include, but are not limited to, gas storage vessels (for example, an article having an open end, a closed end, and a cylindrical portion between, an article having an open end and a spherical and/or round portion, such as, a gas cylinder or an air can), fittings (for example, unions, connectors, adaptors, other connections between two or more pieces of tubing, for example, capable of making a leak-free or substantially leak-free seal), compression fittings (including ferrules, such as, a front and back ferrule), tubing (for example, coiled tubing, tubing sections such as used to connect a sampling apparatus, pre-bent tubing, straight tubing, loose wound tubing, tightly bound tubing, and/or flexible tubing, whether consisting of the interior being treated or including the interior and the exterior being treated), valves (such as, gas sampling, liquid sampling, transfer, shut-off, or check valves, for example, including a rupture disc, stem, poppet, rotor, multi-position configuration, able to handle vacuum or pressure, a handle or stem for a knob, ball-stem features, ball valve features, check valve features, springs, multiple bodies, seals, needle valve features, packing washers, and/or stems), quick-connects, sample cylinders, regulators and/or flow-controllers (for example, including o-rings, seals, and/or diaphragms), injection ports (for example, for gas chromatographs), in-line filters (for example, having springs, sintered metal filters, mesh screens, and/or weldments), frits, columns, materials, glass liners, gas chromatograph components, liquid chromatography components, components associated with vacuum systems and chambers, components associated with analytical systems, sample probes, control probes, sampling containers, drilled and/or machined block components, manifolds, particles, powders, or a combination thereof.

In another embodiment, the medical device product or medical device process includes elements from one or more of the following patent applications, each of which are incorporated by reference in their entirety, and the medical device product or medical device process have operational properties outside the limitations such following patent applications:

| Title | Publication Number |
|---|---|
| Methods for Culturing Cells in an Alternating Ionic Magnetic Resonance | US20200017848A1 |
| (AIMR) Multiple-Chambered Culture Apparatus | |
| POROUS MATERIALS, METHODS OF MAKING AND USES | US20200038550A1 |
| Prosthetic implantable antibacterial surgical mesh | US10688224B1 |
| Mass Colored Polymer Composition For Use In Medical Technology | US20190338116A1 |
| Applications | |
| AEROSOL DELIVERY SYSTEMS, COMPOSITIONS AND METHODS | US20200054558A1 |
| Graphene Field Effect Transistors for Detection of Ions | US20190293595A1 |
| METHOD OF DRUG DELIVERY FOR PTH, PTHrP AND RELATED PEPTIDES | US20190290738A1 |
| BI-SPECIFIC ANTIGEN-BINDING POLYPEPTIDES | US20190248918A1 |
| Interrogator and interrogation system employing the same | US10628645B2 |
| Interrogator and Interrogation System Employing the Same | US20190213365A1 |
| COMPOUNDS AND METHODS FOR TREATMENT OF CYSTIC FIBROSIS | US20190210973A1 |
| SYSTEMS AND METHODS FOR TREATING SHOULDER PAIN RELATED TO | US20190151650A1 |
| SUBACROMIAL IMPINGEMENT SYNDROME | |
| PARTICLES FOR USE IN ACOUSTIC STANDING WAVE PROCESSES | US20190144813A1 |
| DEVICES AND SYSTEMS FOR CHROMATOGRAPHY COLUMN BED SUPPORT | US20190178854A1 |
| MANAGEMENT AND RELATED METHODS | |
| ANTIBODIES AGAINST THE ECTODOMAIN OF ERBB3 AND USES THEREOF | US20190292271A1 |
| METHODS AND COMPOSITIONS FOR TREATING CYSTIC FIBROSIS | US20200261483A1 |
| METHODS FOR TREATING BALDNESS AND PROMOTING HAIR GROWTH | US20190381296A1 |
| HER2/neu-Specific Antibodies and Methods of Using Same | US20190233539A1 |
| SPECULUM SLEEVE | US20190082948A1 |
| ENGINEERED SCAFFOLDS FOR VASCULARIZED TISSUE REPAIR | US20190247539A1 |
| METHODS OF TREATMENT FOR CYSTIC FIBROSIS | US20200171015A1 |
| ELECTROSPRAY CATHETER | US20200215307A1 |
| SUBPOPULATIONS OF SPORE-LIKE CELLS AND USES THEREOF | US20190060373A1 |
| Interrogator and interrogation system employing the same | US10482293B2 |
| DIAGNOSTIC BIOMARKERS FOR FIBROTIC DISORDERS | US20180280476A1 |
| INTERROGATOR AND INTERROGATION SYSTEM EMPLOYING THE SAME | US20180285606A1 |
| Antibodies with Altered Binding to FcRn and Methods of Using Same | US20190010243A1 |
| DEVICES AND METHODS FOR TREATING A VESSEL IN A SUBJECT | US20190350567A1 |
| METHODS AND COMPOSITIONS FOR TREATING AND DIAGNOSING | US20200061176A1 |
| AUTOIMMUNE DISEASES | |
| EXPANSION OF TUMOR INFILTRATING LYMPHOCYTES FROM LIQUID | US20200224161A1 |
| TUMORS AND THERAPEUTIC USES THEREOF | |
| Acoustic perfusion devices | US10704021B2 |
| ACOUSTIC PERFUSION DEVICES | US20180298323A1 |
| METHODS OF TREATING CYSTIC FIBROSIS IN PATIENTS WITH RESIDUAL | US20180280349A1 |
| FUNCTION MUTATIONS | |
| SYSTEM AND METHOD FOR PACKAGING AN ELONGATE MEDICAL DEVICE | US20180214665A1 |
| ANTI-FGFR ANTIBODIES AND METHODS OF USE | US20180244785A1 |
| EXPANSION OF TUMOR INFILTRATING LYMPHOCYTES (TILS) WITH TUMOR | US20200121719A1 |
| NECROSIS FACTOR RECEPTOR SUPERFAMILY (TNFRSF) AGONISTS AND | |
| THERAPEUTIC COMBINATIONS OF TILS AND TNFRSF AGONISTS | |
| ORAL DEVICE AND METHOD FOR THE USE THEREOF | US20190307638A1 |
| Systems and methods for treating shoulder pain related to subacromial | US10166384B2 |
| impingement syndrome | |
| SYSTEMS AND METHODS FOR TREATING SHOULDER PAIN RELATED TO | US20180078756A1 |
| SUBACROMIAL IMPINGEMENT SYNDROME | |
| Protein-protein interaction as biomarkers | US10139410B2 |
| PROTEIN-PROTEIN INTERACTION AS BIOMARKERS | US20180156798A1 |
| ANTIMICROBIAL WOUND CARE DRESSING | US20190247234A1 |
| Catheter-based system for delivery and retrieval of a leadless pacemaker | US10448973B2 |
| Catheter-based system for delivery and retrieval of a leadless pacemaker | US10575872B2 |
| Catheter-based system for delivery and retrieval of a leadless pacemaker | US10743916B2 |
| CATHETER-BASED SYSTEM FOR DELIVERY AND RETRIEVAL OF A LEADLESS | US20180104449A1 |
| PACEMAKER | |
| CATHETER-BASED SYSTEM FOR DELIVERY AND RETRIEVAL OF A LEADLESS | US20180104450A1 |
| PACEMAKER | |
| CATHETER-BASED SYSTEM FOR DELIVERY AND RETRIEVAL OF A LEADLESS | US20180104451A1 |
| PACEMAKER | |
| CATHETER-BASED SYSTEM FOR DELIVERY AND RETRIEVAL OF A LEADLESS | US20180104452A1 |
| PACEMAKER | |
| CATHETER-BASED SYSTEM FOR DELIVERY AND RETRIEVAL OF A LEADLESS | US20180303513A1 |
| PACEMAKER | |
| CATHETER-BASED SYSTEM FOR DELIVERY AND RETRIEVAL OF A LEADLESS | US20180303514A1 |
| PACEMAKER | |
| Acoustic perfusion devices | US10662402B2 |
| ACOUSTIC PERFUSION DEVICES | US20180010085A1 |
| IMPLANT APPLICATORS | US20190374380A1 |
| ACOUSTIC PERFUSION DEVICES | US20180066224A1 |
| OCCLUSION-RESISTANT CATHETER WITH OCCLUSION-RESISTANT TIP | US20190201662A1 |
| Interrogator and interrogation system employing the same | US10185853B2 |
| Interrogator and Interrogation System Employing the Same | US20170316236A1 |
| Alternating Ionic Magnetic Resonance (AIMR) Multiple-Chambered Culture | US20170298340A1 |
| Apparatus | |
| Methods for Culturing Cells in an Alternating Ionic Magnetic Resonance | US20170298341A1 |
| (AIMR) Multiple-Chambered Culture Apparatus | |
| ANTIBODIES AGAINST EPIDERMAL GROWTH FACTOR RECEPTOR (EGFR) AND | US20170314079A1 |
| USES THEREOF | |
| Methods for the treatment of disease using immunoglobulins having Fc | US10711069B2 |
| regions with altered affinities for FcγRactivating and FeγRinhibiting | |
| Methods for the Treatment of Disease Using Immunoglobulins Having Fc | US20180009902A1 |
| Regions with Altered Affinities for FcgammaRactivating and | |
| FcgammaRinhibiting | |
| BCR-complex-specific antibodies and methods of using same | US10479831B2 |
| BCR-Complex-Specific Antibodies and Methods of Using Same | US20180051079A1 |
| Oral Examination Device | US20170333294A1 |
| INTERROGATOR AND INTERROGATION SYSTEM EMPLOYING THE SAME | US20170243033A1 |
| Interrogator and interrogation system employing the same | U.S. Pat. No. 9,996,717B2 |
| Embryonic Cell-Based Therapeutic Candidate Screening Systems, Models for | US20190195863A1 |
| Huntington's Disease and Uses Thereof | |
| DEUTERIUM-MODIFIED CFTR MODULATORS | US20190048020A1 |
| ACOUSTIC PERFUSION DEVICES | US20170137774A1 |
| Acoustic perfusion devices | U.S. Pat. No. 9,745,548B2 |
| Cellular or Viral Membrane Coated Nanostructures and Uses Thereof | US20200268892A1 |
| ORAL DEVICE, ASSEMBLY AND METHOD FOR THE USE THEREOF | US20180360692A1 |
| Devices and Dispensers for Sterile Coverings for Tablet Computers and | US20170085287A1 |
| Mobile Phones | |
| CHAMBER CUSHION, SEAL AND USE THEREOF | US20170143577A1 |
| METHOD OF DRUG DELIVERY FOR PTH, PTHrP AND RELATED PEPTIDES | US20170189493A1 |
| RELIABLE DEPOSITION OF THIN PARYLENE | US20170113246A1 |
| ln-situ heated disposition of parylene to enhance pore penetration into | US10287451B2 |
| silicone | |
| IN-SITU HEATED DEPOSITION OF PARYLENE TO ENHANCE PORE | US20170009097A1 |
| PENETRATION INTO SILICONE | |
| ANTIBODIES AGAINST THE ECTODOMAIN OF ERBB3 AND USES THEREOF | US20170073427A1 |
| METHODS AND COMPOSITIONS FOR TAUOPATHY DIAGNOSIS AND | US20190234966A1 |
| TREATMENT | |
| SUBPOPULATIONS OF SPORE-LIKE CELLS AND USES THEREOF | US20170007647A1 |
| Subpopulations of spore-like cells and uses thereof | U.S. Pat. No. 9,999,638B2 |
| HER2/neu-specific antibodies and methods of using same | US10131713B2 |
| HER2/neu-Specific Antibodies and Methods of Using Same | US20170218083A1 |
| LOADING SYSTEM FOR AN ENCAPSULATION DEVICE | US20160374900A1 |
| PARYLENE-BASED MICROELECTRODE ARRAY IMPLANT FOR SPINAL CORD | US20170157389A1 |
| STIMULATION | |
| Lubricious Silicone Cable Jackets and Cable Assemblies Formed Therefrom | US20180023248A1 |
| SYSTEMS AND METHODS FOR TREATING SHOULDER PAIN RELATED TO | US20170056645A1 |
| SUBACROMIAL IMPINGEMENT SYNDROME | |
| Systems and methods for treating shoulder pain related to subacromial | U.S. Pat. No. 9,827,412B2 |
| impingement syndrome | |
| ULTRATHIN PARYLENE-C SEMIPERMEABLE MEMBRANES FOR BIOMEDICAL | US20160361463A1 |
| APPLICATIONS | |
| LIQUID APPLICATOR | US20190117208A1 |
| Jaw structure with distal closure for electrosurgical instruments | US10485607B2 |
| Non-linear jaw gap for electrosurgical instruments | US10646269B2 |
| Jaw structure with distal post for electrosurgical instruments | US10702329B2 |
| JAW STRUCTURE WITH DISTAL POST FOR ELECTROSURGICAL INSTRUMENTS | US20170312014A1 |
| NON-LINEAR JAW GAP FOR ELECTROSURGICAL INSTRUMENTS | US20170312015A1 |
| JAW STRUCTURE WITH DISTAL CLOSURE FOR ELECTROSURGICAL | US20170312016A1 |
| INSTRUMENTS | |
| ELECTROSURGICAL INSTRUMENT WITH ELECTRICALLY CONDUCTIVE GAP | US20170312017A1 |
| SETTING MEMBER AND ELECTRICALLY INSULATIVE TISSUE ENGAGING | |
| MEMBERS | |
| ELECTROSURGICAL INSTRUMENT WITH CONDUCTIVE GAP SETTING | US20170312018A1 |
| MEMBER AND INSULATIVE TISSUE ENGAGING MEMBER HAVING VARIABLE | |
| DIMENSIONS AND STIFFNESS | |
| ELECTROSURGICAL INSTRUMENT WITH ELECTRICALLY CONDUCTIVE GAP | US20170312019A1 |
| SETTING AND TISSUE ENGAGING MEMBERS | |
| ACOUSTIC PERFUSION DEVICES | US20160237394A1 |
| ACOUSTIC PERFUSION DEVICES | US20160237395A1 |
| Acoustic perfusion devices | U.S. Pat. No. 9,738,866B2 |
| Acoustic perfusion devices | U.S. Pat. No. 9,752,113B2 |
| WOUND DRESSING | US20180071428A1 |
| Antibodies with Altered Binding to FcRn and Methods of Using Same | US20160289333A1 |
| HER2/neu-Specific Antibodies and Methods of Using Same | US20160257761A1 |
| NANOBODY DIMERS LINKED VIA C-TERMINALLY ENGINEERED CYSTEINS | US20190100599A1 |
| HER2/neu-Specific Antibodies and Methods of Using Same | US20160130360A1 |
| HER2/neu-specific antibodies and methods of using same | U.S. Pat. No. 9,469,692B2 |
| Piezoelectric film sensor, piezoelectric film sensor circuit and methods for | US10036675B2 |
| manufacturing the same | |
| PIEZOELECTRIC FILM SENSOR, PIEZOELECTRIC FILM SENSOR CIRCUIT AND | US20170194427A1 |
| METHODS FOR MANUFACTURING THE SAME | |
| ACOUSTIC PERFUSION DEVICES | US20160102284A1 |
| CYSTEINE LINKED NANOBODY DIMERS | US20170360955A1 |
| Acoustic perfusion devices | U.S. Pat. No. 9,822,333B2 |
| METHOD FOR CONNECTING TWO OBJECTS USING A POLYMER | US20180320026A1 |
| COMPOSITION AND A SYSTEM, FOR USE IN A COSMETIC PROCEDURE | |
| ULTRA-HIGH DENSITY CELL BANKING METHODS | US20160100570A1 |
| Antibodies against epidermal growth factor receptor (EGFR) and uses | US10072299B2 |
| thereof | |
| ANTIBODIES AGAINST EPIDERMAL GROWTH FACTOR RECEPTOR (EGFR) AND | US20150368346A1 |
| USES THEREOF | |
| ANTIBODIES AGAINST EPIDERMAL GROWTH FACTOR RECEPTOR (EGFR) AND | US20150368347A1 |
| USES THEREOF | |
| ANTIBODIES AGAINST EPIDERMAL GROWTH FACTOR RECEPTOR (EGFR) AND | US20150368361A1 |
| USES THEREOF | |
| ANTIBODIES AGAINST EPIDERMAL GROWTH FACTOR RECEPTOR (EGFR) AND | US20150376284A1 |
| USES THEREOF | |
| Antibodies against epidermal growth factor receptor (EGFR) and uses | U.S. Pat. No. 9,885,087B2 |
| thereof | |
| Antibodies against epidermal growth factor receptor (EGFR) and uses | U.S. Pat. No. 9,902,999B2 |
| thereof | |
| Subpopulations of Spore-Like Cells and Uses Thereof | US20150376576A1 |
| Subpopulations of spore-like cells and uses thereof | U.S. Pat. No. 9,663,765B2 |
| Porous materials, methods of making and uses | US10391199B2 |
| POROUS MATERIALS, METHODS OF MAKING AND USES | US20160022866A1 |
| Regulation of autonomic control of bladder voiding after a complete spinal | US10751533B2 |
| cord injury | |
| REGULATION OF AUTONOMIC CONTROL OF BLADDER VOIDING AFTER A | US20170274209A1 |
| COMPLETE SPINAL CORD INJURY | |
| Implantable chamber for collection and supply of interstitial fluid | US10342961B2 |
| BALLOON FOR MEDICAL DEVICE | US20160346522A1 |
| ANTIBODIES AGAINST EPIDERMAL GROWTH FACTOR RECEPTOR (EGFR) AND | US20160002339A1 |
| USES THEREOF | |
| Low resource method and device for detecting analytes | US10578615B2 |
| LOW RESOURCE METHOD AND DEVICE FOR DETECTING ANALYTES | US20170030903A1 |
| Bottles with means to prevent gushing | US10011391B2 |
| BOTTLES WITH MEANS TO PREVENT GUSHING | US20150197371A1 |
| Use of aliginate oligomers and CFTR modulators in treatment of conditions | US10751363B2 |
| associated with CFTR dysfunction | |
| USE OF ALGINATE OLIGOMERS AND CFTR MODULATORS IN THE | US20180055873A1 |
| TREATMENT OF CONDITIONS ASSOCIATED WITH CFTR DYSFUNCTION | |
| Bi-specific antigen-binding polypeptides | US10202462B2 |
| BI-SPECIFIC ANTIGEN-BINDING POLYPEPTIDES | US20150299335A1 |
| Micro-needle device | US10675452B2 |
| MICRO-NEEDLE DEVICE | US20170173316A1 |
| BCR-Complex-Specific Antibodies and Methods of Using Same | US20150166658A1 |
| BCR-complex-specific antibodies and methods of using same | U.S. Pat. No. 9,695,236B2 |
| Carbon nano tube based antennas | US10485482B2 |
| CARBON NANO TUBE BASED ANTENNAS | US20170165471A1 |
| Interrogator and Interrogation System Employing the Same | US20150169918A1 |
| Interrogator and interrogation system employing the same | U.S. Pat. No. 9,710,683B2 |
| AEROSOL DELIVERY SYSTEMS, COMPOSITIONS AND METHODS | US20150150803A1 |
| Hemostatic compositions, assemblies, systems, and methods employing | US20150125513A1 |
| particulate hemostatic agents formed from hydrophilic polymer foam such | |
| as chitosan | |
| Hemostatic compositions, assemblies, systems, and methods employing | U.S. Pat. No. 9,132,206B2 |
| particulate hemostatic agents formed from hydrophilic polymer foam such | |
| as chitosan | |
| Bridle catheter | US10314994B2 |
| Bridle Catheter | US20150157828A1 |
| SYSTEMS AND METHODS FOR TREATING SHOULDER PAIN RELATED TO | US20150134037A1 |
| SUBACROMIAL IMPINGEMENT SYNDROME | |
| Systems and methods for treating shoulder pain related to subacromial | U.S. Pat. No. 9,381,343B2 |
| impingement syndrome | |
| POROUS MATERIALS, METHODS OF MAKING AND USES | US20150028510A1 |
| Porous materials, methods of making and uses | U.S. Pat. No. 9,522,502B2 |
| IN-SITU HEATED DEPOSITION OF PARYLENE TO ENHANCE PORE | US20150111023A1 |
| PENETRATION INTO SILICONE | |
| ln-situ heated deposition of parylene to enhance pore penetration into | U.S. Pat. No. 9,469,778B2 |
| silicone | |
| TWEAK ANTAGONISTS FOR TREATING LUPUS NEPHRITIS AND MUSCLE | US20160243224A1 |
| ATROPHY | |
| ULTRATHIN PARYLENE-C SEMIPERMEABLE MEMBRANES FOR BIOMEDICAL | US20150147810A1 |
| APPLICATIONS | |
| HER2/neu-Specific Antibodies and Methods of Using Same | US20140328836A1 |
| HER2/neu-specific antibodies and methods of using the same | U.S. Pat. No. 9,243,069B2 |
| PARYLENE-BASED MICROELECTRODE ARRAY IMPLANT FOR SPINAL CORD | US20140316503A1 |
| STIMULATION | |
| Method of constructing an implantable microelectrode array | U.S. Pat. No. 9,409,011B2 |
| Antibodies to Plasminogen Activator lnhibitor-1 (PAI-1) and Uses Thereof | US20160200831A1 |
| Antibodies to Plasminogen Activator lnhibitor-1 (PAI-1) and Uses Thereof | US20180155444A1 |
| Antibodies to Plasminogen Activator lnhibitor-1 (PAI-1) and Uses Thereof | US20190359729A1 |
| Antibodies to plasminogen activator inhibitor-1 (PAI-1) and uses thereof | U.S. Pat. No. 9,845,363B2 |
| RECEPTORS FOR B7-H4 | US20160146806A1 |
| NEW TREATMENT | US20170112839A1 |
| PROTECTIVE COATINGS INCLUDING PARTIALLY HALOGENATED MATERIALS | US20170073526A1 |
| AND METHODS FOR FORMING AND USING SAME | |
| ANTIBODIES AGAINST EPIDERMAL GROWTH FACTOR RECEPTOR (EGFR) AND | US20140234314A1 |
| USES THEREOF | |
| PARY-XYLENE BASED MICROFILM ELUTION DEVICES | US20140236080A1 |
| Pary-xylene based microfilm elution devices | U.S. Pat. No. 9,040,071B2 |
| Hemostatic compositions, assemblies, systems, and methods employing | US20140228879A1 |
| particulate hemostatic agents formed from hydrophilic polymer foam such | |
| as chitosan | |
| Hemostatic compositions, assemblies, systems, and methods employing | U.S. Pat. No. 8,951,565B2 |
| particulate hemostatic agents formed from hydrophilic polymer foam such | |
| as chitosan | |
| POROUS MATERIALS, METHODS OF MAKING AND USES | US20140179819A1 |
| IMPLANTABLE CHAMBER FOR COLLECTION AND SUPPLY OF INTERSTITIAL | US20170007808A1 |
| FLUID | |
| IMPLANTABLE CHAMBER FOR COLLECTION AND SUPPLY OF INTERSTITIAL | US20180056049A9 |
| FLUID | |
| ANTIBODIES AGAINST THE ECTODOMAIN OF ERBB3 AND USES THEREOF | US20140234329A1 |
| Antibodies against the ectodomain of ERBB3 and uses thereof | U.S. Pat. No. 9,487,588B2 |
| Methods for the Treatment of Disease Using Immunoglobulins Having Fc | US20140134162A1 |
| Regions with Altered Affinities for FcgammaRactivating and | |
| FcgammaRinhibiting | |
| Methods for the treatment of disease using immunoglobulins having Fc | U.S. Pat. No. 9,708,408B2 |
| Regions with altered affinities for FcγRactivating and FcγRinhibiting | |
| BACKFLOW PREVENTER VALVE | US20140137949A1 |
| Backflow preventer valve | U.S. Pat. No. 9,587,752B2 |
| ANTIBODIES AGAINST EPIDERMAL GROWTH FACTOR RECEPTOR (EGFR) AND | US20140127207A1 |
| USES THEREOF | |
| Antibodies against epidermal growth factor receptor (EGFR) and uses | U.S. Pat. No. 9,226,964B2 |
| thereof | |
| NASAL CANNULA FOR DELIVERY OF AEROSOLIZED MEDICAMENTS | US20140158127A1 |
| COMPOSITIONSAND METHODS FOR TREATING PROTEINOPATHIES | US20150284472A1 |
| INTERROGATOR AND INTERROGATION SYSTEM EMPLOYING THE SAME | US20140028444A1 |
| Interrogator and interrogation system employing the same | U.S. Pat. No. 9,646,182B2 |
| BCR-Complex-Specific Antibodies and Methods of Using Same | US20140017237A1 |
| BCR-complex-specific antibodies and methods of using same | U.S. Pat. No. 8,993,730B2 |
| Subpopulations of Spore-Like Cells and Uses Thereof | US20140017708A1 |
| Subpopulations of spore-like cells and uses thereof | U.S. Pat. No.9,145,545B2 |
| PACKAGING SYSTEM FOR PRESERVING A NONOXYGENATED HEMOGLOBIN | US20130327677A1 |
| BASED OXYGEN THERAPEUTIC PRODUCT | |
| Packaging system for preserving a nonoxygenated hemoglobin based | U.S. Pat. No. 9,174,771B2 |
| oxygen therapeutic product | |
| LIGHT CURE BITE PLATE FOR ORTHODONTIC REMODELING DEVICES | US20140080082A1 |
| Device for explorable stomach gastric bypass | US10070979B2 |
| DEVICE FOR EXPLORABLE STOMACH GASTRIC BYPASS | US20160193062A1 |
| DEVICE FOR EXPLORABLE STOMACH GASTRIC BYPASS | US20180344499A1 |
| BIOCOMPATIBLE AND BIOSTALE IMPLANTABLE MEDICAL DEVICE | US20130317288A1 |
| PARYLENE-BASED MICROELECTRODE ARRAY IMPLANT FOR SPINAL CORD | US20130310911A1 |
| STIMULATION | |
| Parylene-based microelectrode array implant for spinal cord stimulation | U.S. Pat. No. 8,805,542B2 |
| COMPOSITION FOR LUPUS NEPHRITIS AND METHODS OF MAKING AND | US20130345257A1 |
| USING THE SAME | |
| BIOCOMPATIBLE AND BIOSTABLE IMPLANTABLE MEDICAL DEVICE | US20130281774A1 |
| HEPATITIS C VIRUS NEUTRALIZING ANTIBODY | US20150118242A1 |
| Hepatitis C virus neutralizing antibody | U.S. Pat. No. 9,399,672B2 |
| Targeting o-acetylated gd2 ganglioside as a new therapeutic and diagnostic | US10745489B2 |
| strategy for Cancer Stem Cells cancer | |
| TARGETING O-ACETYLATED GD2 GANGLIOSIDE AS A NEW THERAPEUTIC | US20190023808A1 |
| AND DIAGNOSTIC STRATEGY FOR CANCER STEM CELLS CANCER | |
| Methods for Culturing Cells in an Alternating Ionic Magnetic Resonance | US20130267003A1 |
| (AIMR) Multiple-Chambered Culture Apparatus | |
| Alternating Ionic Magnetic Resonance (AIMR) Multiple-Chambered Culture | US20130267020A1 |
| Apparatus | |
| Alternating ionic magnetic resonance (AIMR) multiple-chambered culture | U.S. Pat. No. 9,914,920B2 |
| apparatus | |
| Diagnostic BioMarkers for Fibrotic Disorders | US20130209490A1 |
| ORAL DEVICE AND METHOD FOR THE USE THEREOF | US20130296751A1 |
| Diagnostic BioMarkers for Fibrotic Disorders | US20160228509A9 |
| Oral device and method for the use thereof | U.S. Pat. No. 9,855,187B2 |
| AEROSOL DELIVERY SYSTEMS, COMPOSITIONS AND METHODS | US20140109899A1 |
| Aerosol delivery systems, compositions and methods | U.S. Pat. No. 8,945,605B2 |
| Appliance for an Electromagnetic Spectrum Sensor Monitoring an | US20130237832A1 |
| Intravascular Infusion | |
| Appliance for an Electromagnetic Spectrum Sensor Monitoring an | US20130237833A1 |
| Intravascular Infusion | |
| Dressing for an Electromagnetic Spectrum Sensor Monitoring an | US20130237834A1 |
| Intravascular Infusion | |
| Dressing for an Electromagnetic Spectrum Sensor Monitoring an | US20130237835A1 |
| Intravascular Infusion | |
| Dressing for an Electromagnetic Spectrum Sensor Monitoring an | US20130237836A1 |
| Intravascular Infusion | |
| Method of Using an Electromagnetic Spectrum Sensor to Monitor an | US20130237837A1 |
| Intravascular Infusion | |
| Systems and Methods to Mitigate the Effects of Skin Moisture on a | US20130237779A1 |
| Percutaneous Infrared Signal | |
| Appliance for an Electromagnetic Spectrum Sensor Monitoring an | US20130237812A1 |
| Intravascular Infusion | |
| Appliance for an Electromagnetic Spectrum Sensor Monitoring an | US20130237830A1 |
| Intravascular Infusion | |
| Appliance for an Electromagnetic Spectrum Sensor Monitoring an | US20130237831A1 |
| Intravascular Infusion | |
| Method Of Drug Delivery For PTH, PTHrP And Related Peptides | US20140046292A1 |
| Method Of Drug Delivery For PTH, PTHrP And Related Peptides | US20140046293A1 |
| Nucleic acids encoding antibodies to Kallidin and des-Arg10-Kallidin and | US10465002B2 |
| host cells comprising the same | |
| ANTIBODIES TO BRADYKININ B1 RECEPTOR LIGANDS | US20150050270A1 |
| ANTIBODIES TO KALLIDIN AND DES-ARG9-KALLIDIN | US20160368976A1 |
| METHODS FOR TREATING PAIN USING ANTIBODIES TO KALLIDIN AND DES- | US20180222967A1 |
| ARG10-KALLIDIN | |
| METHODS FOR TREATING INFLAMMATION USING ANTIBODIES TO KALLIDIN | US20200115441A1 |
| AND DES-ARG10-KALLIDIN | |
| Antibodies to kallidin and des-Arg9-kallidin | U.S. Pat. No. 9,376,494B2 |
| Methods for treating pain using antibodies to Kallidin and des-Arg10- | U.S. Pat. No. 9,879,079B2 |
| Kallidin | |
| Electrospun Porous Media | US20130197664A1 |
| Individualized vaccines for cancer | US10155031B2 |
| INDIVIDUALIZED VACCINES FOR CANCER | US20160058853A1 |
| Individualized Vaccines for Cancer | US20190083593A1 |
| LINER-BASED SHIPPING AND DISPENSING CONTAINERS FOR THE | US20140231427A1 |
| SUBSTANTIALLY STERILE STORAGE, SHIPMENT, AND DISPENSE OF | |
| MATERIALS | |
| PROTEIN-PROTEIN INTERACTION AS BIOMARKERS | US20140256649A1 |
| Protein-protein interaction as biomarkers | U.S. Pat. No. 9,804,160B2 |
| POROUS MATERIALS, METHODS OF MAKING AND USES | US20130023987A1 |
| Porous materials, methods of making and uses | U.S. Pat. No. 9,138,309B2 |
| SILICONE COATED IMPLANT | US20130236524A1 |
| FIXER OCCLUDER PENILE DEVICE FOR URINARY INCONTINENCE | US20140041672A1 |
| SYSTEMS AND METHODS FOR PERCUTANEOUS ELECTRICAL STIMULATION | US20130197615A1 |
| MICROSTRUCTURE DEVICE WITH REMOVABLE MICROSTRUCTURE PATCH | US20140207101A1 |
| MICROELECTRODE WITH LATERALLY EXTENDING PLATFORM FOR | US20120323103A1 |
| REDUCTION OF TISSUE ENCAPSULATION | |
| Microelectrode with laterally extending platform for reduction of tissue | U.S. Pat. No. 8,463,353B2 |
| encapsulation | |
| METHOD OF DRUG DELIVERY FOR PTH, PTHrP AND RELATED PEPTIDES | US20130006217A1 |
| DEVICES AND DISPENSERS FOR STERILE COVERINGS FOR TABLET | US20130186798A1 |
| COMPUTERS AND MOBILE PHONES | |
| Devices and dispensers for sterile coverings for tablet computers and | U.S. Pat. No. 9,550,620B2 |
| mobile phones | |
| DEVICES AND DISPENSERS FOR STERILE COVERINGS FOR TABLET | US20130188890A1 |
| COMPUTERS AND MOBILE PHONES | |
| ULTRATHIN PARYLENE-C SEMIPERMEABLE MEMBRANES FOR BIOMEDICAL | US20130143326A1 |
| APPLICATIONS | |
| Ultrathin parylene-C semipermeable membranes for biomedical | U.S. Pat. No. 8,877,489B2 |
| applications | |
| Methods For Treating Baldness And Promoting Hair Growth | US20140079686A1 |
| SYSTEM AND METHOD FOR TREATING SHOULDER PAIN RELATED TO | US20140046416A1 |
| SUBACROMIAL IMPINGEMENT SYNDROME | |
| System and method for treating shoulder pain related to subacromial | U.S. Pat. No. 8,886,337B2 |
| impingement syndrome | |
| PARYLENE-C AS A PIEZOELECTRIC MATERIAL AND METHOD TO MAKE IT | US20120091858A1 |
| Parylene-C as a piezoelectric material and method to make it | U.S. Pat. No. 8,732,922B2 |
| POROUS MATERIALS, METHODS OF MAKING AND USES | US20120077012A1 |
| Porous materials, methods of making and uses | U.S. Pat. No. 8,889,751B2 |
| SCREENING SYSTEMS UTILIZING RTP801 | US20120034599A1 |
| Screening systems utilizing RTP801 | U.S. Pat. No.8,344,104B2 |
| OPEN-CELL SURFACE FOAM MATERIALS | US20110309541A1 |
| Individualized vaccines for cancer | US10738355B2 |
| INDIVIDUALIZED VACCINES FOR CANCER | US20140178438A1 |
| BIOCOMPATIBLE AND BIOSTABLE IMPLANTABLE MEDICAL DEVICE | US20110270028A1 |
| POROUS MATERIALS, METHODS OF MAKING AND USES | US20110276133A1 |
| POROUS MATERIALS, METHODS OF MAKING AND USES | US20110282444A1 |
| ANTIBODIES AGAINST EPIDERMAL GROWTH FACTOR RECEPTOR (EGFR) AND | US20110287002A1 |
| USES THEREOF | |
| Antibodies against epidermal growth factor receptor (EGFR) and uses | U.S. Pat. No.9,044,460B2 |
| thereof | |
| Compositions and Methods to Treat Cardiac Pacing Conditions | US20110300107A1 |
| Systems and Methods to Treat Cardiac Pacing Conditions | US20110301568A1 |
| Compositions to Treat Cardiac Pacing Conditions | US20110319478A1 |
| Methods for treating cardiac pacing conditions | U.S. Pat. No.8,734,780B2 |
| MEDICAL AND DENTAL BIOMATERIAL AND METHOD OF USE FOR THE SAME | US20110182995A1 |
| Medical and dental biomaterial and method of use for the same | U.S. Pat. No.8,105,086B2 |
| Insufflation apparatus | U.S. Pat. No.9,381,312B1 |
| LOADING SYSTEM FOR AN ENCAPSULATION DEVICE | US20140014226A1 |
| Loading system for an encapsulation device | U.S. Pat. No.9,433,557B2 |
| METHODS AND COMPOUNDS FOR MUSCLE GROWTH | US20120296403A1 |
| COMPOSITIONS AND METHODS FOR REDUCING THE MUTATION RATE OF | US20110206638A1 |
| VIRUSES | |
| DRY POWDER INHALER WITH FLUTTER DISPERSION MEMBER | US20120234322A1 |
| Dry powder inhaler with flutter dispersion member | U.S. Pat. No.9,492,625B2 |
| SUBPOPULATIONS OF SPORE-LIKE CELLS AND USES THEREOF | US20110281282A1 |
| Subpopulations of spore-like cells and uses thereof | U.S. Pat. No.8,551,775B2 |
| ANTIBODIES AGAINST ERBB3 AND USES THEREOF | US20110123523A1 |
| Antibodies against ERBB3 and uses thereof | U.S. Pat. No.8,961,966B2 |
| Fc Region-Containing Polypeptides That Exhibit Improved Effector Function | US20120219551A1 |
| Due To Alterations Of The Extent Of Fucosylation, And Methods For Their | |
| Use | |
| Fc region-containing polypeptides that exhibit improved effector function | U.S. Pat. No.9,096,877B2 |
| due to alterations of the extent of fucosylation, and methods for their use | |
| BIOCOMPATIBLE AND BIOSTABLE IMPLANTABLE MEDICAL DEVICE | US20110270022A1 |

While the invention has been described with reference to one or more embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. In addition, all numerical values identified in the detailed description shall be interpreted as though the precise and approximate values are both expressly identified.

What is claimed is:

1. A medical device product having a coating with NAMSA Class VI certification and properties corresponding to the prior art coatings within U.S. Pat. No. 10,604,660.

2. The medical device product of claim 1, wherein the coating is on a fitting.

3. The medical device product of claim 1, wherein the coating is on tubing.

4. The medical device product of claim 1, wherein the coating is on a valve.

5. The medical device product of claim 1, wherein the coating is on a filter.

6. The medical device product of claim 1, wherein the coating is on a frit.

7. The medical device product of claim 1, wherein the coating is on a probe.

8. The medical device product of claim 1, wherein the coating is on metal.

9. The medical device product of claim 1, wherein the coating is on glass.

10. The medical device product of claim 1, wherein the medical device is a therapeutic device.

11. The medical device product of claim 1, wherein the medical device is a wound care device.

12. The medical device product of claim 1, wherein the medical device is a catheter.

13. The medical device product of claim 1, wherein the medical device is an interrogator.

14. The medical device product of claim 1, wherein the medical device is positioned within a human.

15. The medical device product of claim 1, wherein the medical device is a surgical instrument.

16. The medical device product of claim 1, wherein the medical device is a perfusion device.

17. The medical device product of claim 1, wherein the medical device is a gastric bypass device.

18. The medical device product of claim 1, wherein the medical device is a drug delivery device.

19. The medical device product of claim 1, wherein antibodies are in contact with the coating.

20. A medical device process uses a medical device product having a coating with NAMSA Class VI certification and properties corresponding to the prior art coatings within U.S. Pat. No. 10,604,660.
